(19) United States
(12) Patent Application Publication
(10) Pub. No.: US 2007/0020218 A1
(43) Pub. Date: Jan. 25, 2007

(54) Composition and method for the treatment of psoriasis

(76) Inventor: Eileen Richardson, Encinitas, CA (US)

Correspondence Address: PERKINS COIE LLP, POST OFFICE BOX 1208, SEATTLE, WA, 98111-1208, US

(21) Appl. No.: 11/489,740

(22) Filed: Jul. 18, 2006

Related U.S. Application Data
(60) Provisional application No. 60/701,616, filed on Jul. 21, 2005.

(51) Int. Cl. A61K 31/728; A61K 31/7008; A61K 31/198

(52) U.S. Cl. 424/70.13; 514/54; 514/62; 514/564; 514/707

(57) ABSTRACT

Disclosed are compositions for treating psoriasis. One embodiment of the present invention is a composition for treating psoriasis comprising L-lysine, glucosamine, chondroitin and methylsulfonyl methane. Another embodiment of the present invention is a method for the treatment of psoriasis comprising orally administering a composition comprising L-lysine, glucosamine, chondroitin, and methylsulfonyl methane. Another embodiment of the present invention is a system for the treatment of psoriasis comprising one or more packets of one or more tablets for oral consumption, wherein the tablets comprise L-lysine, glucosamine, chondroitin and methylsulfonyl methane.

### CROSS-REFERENCE TO RELATED APPLICATION

The present application claims priority to U.S. Provisional Patent Application No. 60/701,616, filed Jul. 21, 2005, the disclosure of which is incorporated by reference herein in its entirety.

### FIELD OF THE INVENTION

This invention relates to formulations and methods for the treatment of psoriasis.

### BACKGROUND OF THE INVENTION

Each of the references cited herein is incorporated by reference in its entirety. A listing of the references is set forth at the end of the specification.

Psoriasis is a common chronic condition characterized by patches of itchy, scaly, and sometimes inflamed skin. It affects more than 4.5 million people in the United States. Although the ailment is generally mild, it can sometimes be severe, covering large areas of the skin. Its main characteristic is raised red patches typically covered with silver or whitish skin flakes.

Psoriasis is noncontagious and usually strikes between the ages of 10 and 30, though people of any age may be affected. The most common sites for psoriasis are the scalp, elbows, lower back, buttocks, and knees, but it can also affect the toenails and fingernails, leaving them yellowed and pitted.

Psoriasis is not itchy or painful in most cases and for most of the 6 million Americans who suffer from it, it is more of a cosmetic problem. For about 15% of sufferers, however, the rash is so widespread and uncomfortable that performing daily activities becomes difficult. For a small minority, about 5%, psoriasis is accompanied by joint pain and swelling.

Thus, there is a pressing need for compounds for the effective treatment of psoriasis. There is also a need for methods of using these compounds for said treatment.

### SUMMARY OF THE INVENTION

One embodiment of the present invention is a composition for treating psoriasis comprising L-lysine, glucosamine, chondroitin and methylsulfonyl methane.

Another embodiment of the present invention is a method for the treatment of psoriasis comprising orally administering a composition comprising L-lysine, glucosamine, chondroitin, and methylsulfonyl methane.

Another embodiment of the present invention is a system for the treatment of psoriasis comprising one or more packets of one or more tablets for oral consumption, wherein the tablets comprise L-lysine, glucosamine, chondroitin and methylsulfonyl methane.

These and other aspects of the present invention are elucidated further in the detailed description.

### DETAILED DESCRIPTION

In order to fully understand the manner in which the above-recited details and other advantages and objects according to the invention are obtained, a more detailed description of the invention will be rendered by reference to specific embodiments thereof.

One embodiment of the present invention is a composition comprising L-lysine, glucosamine, chondroitin sulfate and methylsulfonyl methane (MSM) for treatment of psoriasis and/or the symptoms associated with psoriasis. Combining said over the counter supplements results in the reduction of the thickness of the psoriasis scales and the reduction of skin and joint inflammation associated with the psoriasis disorder.

The formulation of said embodiment comprises:
L-lysine for tissue repair and healthy blood vessels, and
glucosimine/chrondroitin with MSM to repair the joint and alleviate joint inflammation, as well as rebuilding connective joint and skin tissue.

In one embodiment, the L-lysine may comprise at least 10% by weight of the total weight of the L-lysine, glucosamine, chondroitin and methylsulfonyl methane, the glucosamine may comprise at least 30% by weight of the total weight of the L-lysine, glucosamine, chondroitin and methylsulfonyl methane, the chondroitin may comprise at least 15% by weight of the total weight of the L-lysine, glucosamine, chondroitin and methylsulfonyl methane, and the methylsulfonyl methane may comprise at least 10% by weight of the total weight of the L-lysine, glucosamine, chondroitin and methylsulfonyl methane.

In another embodiment the L-lysine may comprise between about 10% and 30% by weight of the total weight of the L-lysine, glucosamine, chondroitin and methylsulfonyl methane, the glucosamine may comprise between about 30% to 50% by weight of the total weight of the L-lysine, glucosamine, chondroitin and methylsulfonyl methane, the chondroitin may comprise between about 15% to 35% by weight of the total weight of the L-lysine, glucosamine, chondroitin and methylsulfonyl methane and the methylsulfonyl methane may comprise between about 10% to 30% by weight of the total weight of the L-lysine, glucosamine, chondroitin and methylsulfonyl methane.

In another embodiment the L-lysine may comprise between about 15% and 25% by weight of the total weight of the L-lysine, glucosamine, chondroitin and methylsulfonyl methane, the glucosamine may comprise between about 35% to 45% by weight of the total weight of the L-lysine, glucosamine, chondroitin and methylsulfonyl methane, the chondroitin may comprise between about 20% to 30% by weight of the total weight of the L-lysine, glucosamine, chondroitin and methylsulfonyl methane, and the methylsulfonyl methane may comprise between about 15% to 25% by weight of the total weight of the L-lysine, glucosamine, chondroitin and methylsulfonyl methane.

In these or other embodiments of the present invention, the total weight of the L-lysine, glucosamine, chondroitin and methylsulfonyl methane of the composition may be at least about 5000 mg, at least about 6000 mg, between about 6000 mg and 10000 mg, or between about 7000 mg and 9000 mg.

In another embodiment of the present invention, a method for treatment of psoriasis and/or the symptoms associated with psoriasis is described comprising administering a formulation comprising L-lysine, glucosamine, chondroitin sulfate and methylsulfonyl methane. The formulation may be taken initially 3 to 4 times a day until the psoriasis is minimized and then managed with a maintenance program that may include a reduced dosage. For initial treatment, it is advantageous to have the L-Lysine, glucosamine, chondroitin sulfate and MSM present in a patient at some level throughout the day. To achieve this end a patient may take reduced dosages more often throughout the day.

The preferred single dosage for an adult is about:
L-Lysine 1,500 mg
Glucosamine 3,000 mg
Chondroitin sulfate 1,800 mg
MSM 1,500 mg.

The dosage may be administered four times per day in a single pharmaceutically acceptable carrier or in multiple pharmaceutically acceptable carriers.

The dosage may be adjusted depending on the patient, including age weight or severity of the condition to be treated. Further, the dosage may be reduced over time as the severity of the conditions diminishes.

In another embodiment of the present invention a system is disclosed comprising one or more packets of one or more tablets for oral consumption, wherein the tablets comprise L-lysine, glucosamine, chondroitin and methylsulfonyl methane. Each packet may or may not contain the same tablet or tablets and each of the tablets may or may not be the same.

Psoriasis occurs when skin cells reproduce more quickly than usual. Normally, skin cells are created in the lower layers of the skin and take about 28 days to rise through to the surface, where they eventually are shed. For people with psoriasis, this life cycle of the skin cell lasts only eight days. New cells accumulate so quickly that they don't have time to mature and cannot slough off. The skin then becomes red and inflamed, and overlapping patches of white, scaly skin develop.

There are many symptoms and indications of psoriasis. One such symptom is joint pain and arthritis. Osteoarthritis is caused from the deterioration in the cartilage that protects the bone ends. Sudden injuries or an inherited defect may cause faults in the cartilage, but most commonly the deterioration is due to aging, diet, and lifestyle. The once smooth surfaces of the cartilage become rough which results in friction.

For psoriasis and each of the symptoms, I have found that a combination of homeopathic remedies eliminates one or more of the psoriasis conditions. This is even more apparent for psoriasis inflammation at the joints resulting in the body “calming” down its immune response from its stressed state.

L-Lysine

L-lysine is an essential amino acid which cannot be manufactured in the body, hence it is important to include adequate amounts in the diet and in supplement form. L-lysine may help ensure adequate absorption of calcium and assist with the formation of collagen for bone, cartilage and connective tissue. It may also help improve circulation and assist the immune system manufacture antibodies, at the same time controlling acid/alkaline balance.

Lysine may also help maintain nitrogen balance in the body. L-Lysine provides nutritional support for the body's natural defenses and works with other essential amino acids to maintain growth, lean body mass, and the body's store of nitrogen.

One needs about 1 gram of lysine a day to keep in protein balance, but you can consume lysine, pure lysine, a perfectly non toxic substance in food or pills.

Glucosamine/Chondroitin In Combination

Glucosamine is a natural compound that nourishes the connective tissue structures found in cartilage and joint fluid. It is a precursor for joint fluid, ligaments, tendons, membranes and blood vessels. It helps to maintain structural integrity of joints and connective tissues.

Chondroitin is a naturally occurring nutrient found in connective tissue. It is capable of binding water molecules to lubricate, cushion and support joints. It helps to maintain structural integrity of joints and blood vessels. For years, experts stated that oral chondroitin couldn't possibly work, because its molecules are so big that it seemed doubtful that they could be absorbed through the digestive tract. However, in 1995 researchers laid this objection to rest when they found evidence that up to 15% of chondroitin is absorbed intact.

In 2000, a 6-month double-blind placebo-controlled study of combined glucosamine, chondroitin and manganese for the treatment of knee osteoarthritis found evidence of significant improvement in a treated group. (7)

Glucosamine

A double-blind study compared glucosamine sulfate against placebo in 252 people with osteoarthritis of the knee. (8) After 4 weeks, the group that was given glucosamine experienced significantly reduced pain and improved movement, to a greater extent than the improvements seen in the placebo group. (8)

Another double-blind study followed 329 people who were divided into four groups. (9) One group was given the standard anti-arthritis drug piroxicam (Feldene), a second was given glucosamine, a third received both treatments, and the fourth received placebo only. Over 90 days, piroxicam and glucosamine proved equally effective at reducing symptoms. Interestingly, the combination treatment (piroxicam plus glucosamine) didn't produce significantly better results than either treatment taken alone. After 90 days, treatment was stopped and the participants were followed for an additional 60 days. The benefits of piroxicam rapidly disappeared, but the benefits of glucosamine lasted for the full 60 days.

Similar results have been seen in studies that compared glucosamine against ibuprofen for knee arthritis months. (10)

Chondroitin

Double-blind placebo-controlled studies involving a total of several hundred participants suggest that chondroitin can relieve symptoms of osteoarthritis. One study enrolled 85 people with osteoarthritis of the knee. (11) Improved results were seen in 69% of those taking chondroitin sulfate but in only 32% of those taking a placebo. Good results were seen in a 12-month double-blind trial that compared chondroitin against placebo in 104 individuals with arthritis of the knee. (12)

Methylsulfonyl methane (MSM)

MSM is a naturally occurring compound, source of organic sulfur found naturally in the human body. MSM and related compounds provide nearly 85 percent of the sulfur found in all living organisms. Sulfur comprises approximately 0.25 percent of body weight and is present in significant amounts in nerve tissue, muscle tissue, skin, hair and bones.

MSM is a bioavailable form of sulfur, which is necessary for the proper function of the body's skin, hair, nails, connective tissue, amino acid production, immune system, and healthy joints. Sulfur is a macronutrient and must be consumed each day for healthy function and growth of the body. MSM is found in fresh fruit, vegetables, seafood and meats. MSM levels decline with age.

Further, MSM is the major metabolite of DMSO (dimethyl sulfoxide), and is 34% elemental sulfur, crucial in maintaining healthy tissues. MSM provides sulfur for many important functions in the human body.

While sulfur supports many functions, it is also well known for maintaining connective tissue health. Sulfur supports tissue containing significant amounts of collagen and keratin. Sulfur is essential in methionine, cysteine, and serum protein metabolism.

Now widely available in concentrated supplement form, MSM has been much publicized of late as an effective remedy for back pain, arthritis and a host of other disorders. Evidence for its healing potential, however, is currently only word of mouth, because few rigorously controlled scientific studies have yet been done concerning MSM use in humans.

MSM supplements are made from DMSO (dimethyl sulfoxide), an industrial solvent that the FDA has approved for only one use—a bladder disorder called interstitial cystitis. Years ago, enthusiasts hailed DMSO as a remedy for a variety of ailments, particularly arthritis, but the noxious odor it created in users seriously lessened its appeal. It also caused toxic effects in some people. MSM is thought to have many of DMSO's advantages without the smell or toxicity.

For example, MSM appears to inhibit pain impulses that travel along nerve fibers, acting as an analgesic. This property, along with the compound's potential anti-inflammatory actions, are often cited in explaining its use for combating the symptoms of arthritis, fibromyalgia, carpal tunnel syndrome and allergies.

In addition, MSM may reduce muscle spasms, increase blood flow and possibly contribute to the maintenance and repair of cartilage. Studies in rats indicate that MSM may help to delay the growth of certain types of cancerous tumors.

Further, MSM may help to relieve arthritis symptoms. MSM supplements not only help treat osteoarthritis—the degenerative form of arthritis that wears down cartilage over time—but rheumatoid arthritis, lupus and other autoimmune-related conditions as well.

Numerous studies have shown that sulfur levels in arthritic joints are lower than in healthy joints. MSM may help by delivering needed sulfur to the afflicted areas. Once in the joints, exactly how MSM works remains unclear. It may exert an anti-inflammatory, analgesic effect similar to that of aspirin.

It may also help to maintain or repair cartilage, the gel-like substance that cushions joints and that is a key ingredient of connective tissue. In a preliminary, double-blind study of 16 patients with degenerative arthritis, the patients who took 2,250 mg of MSM daily for six weeks reported an 82% reduction in pain on average. (13) Only two of those taking the placebo reported decreased pain—about 20%.

Pharmaceutical Indications

Pharmaceutically acceptable carriers for the formulations disclosed herein or methods to use said formulations may include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Formulations useful in the methods of the present invention comprise L-lysine, glucosamine, chondroitin sulfate and methylsulfonyl methane, one or more pharmaceutically acceptable carriers therefor, and optionally other therapeutic ingredients. The formulations may conveniently be presented in unit dosage form and may be prepared by any methods well known in the pharmaceutical arts. The amount of active ingredient, which can be combined with a carrier material. to produce a single dosage form, will likely vary depending upon the subject being treated and the particular mode of administration. The amount of L-lysine, glucosamine, chondroitin sulfate and/or methylsulfonyl methane that can be combined with a carrier material to produce a pharmaceutically effective dose will generally be an amount of the L-lysine, glucosamine, chondroitin sulfate and/or methylsulfonyl methane which produces a therapeutic effect. Generally, the amount of the entire volume comprised of the L-lysine, glucosamine, chondroitin sulfate and/or methylsulfonyl methane will range from about one percent to about ninety-nine percent of the L-lysine, glucosamine, chondroitin sulfate and/or methylsulfonyl methane composition, preferably from about ten percent to about eighty percent of the L-lysine, glucosamine, chondroitin sulfate and/or methylsulfonyl methane composition.

Formulations suitable for oral administration may be in the form of capsules, cachets, pills, tablets, lozenges, powders, as granules, bolus, electuary, or a paste, as a solution or a suspension in an aqueous or non- aqueous liquid, as an oil-in-water or water-in-oil liquid emulsion, as an elixir or syrup, or as pastilles (using an inert base, such as gelatin and glycerin, or sucrose and acacia), each containing a predetermined amount of L-lysine, glucosamine, chondroitin sulfate and/or methylsulfonyl methane as an active ingredient.

In solid dosage forms for oral administration, such as capsules, tablets, pills, powders, granules and the like, the L-lysine, glucosamine, chondroitin sulfate and/or methylsulfonyl methane is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate, solution retarding agents, such as paraffin, absorption accelerators, such as quaternary ammonium compounds; wetting agents, such as, for example, acetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the L-lysine, glucosamine, chondroitin sulfate and/or methylsulfonyl methane therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release L-lysine, glucosamine, chondroitin sulfate and/or methylsulfonyl methane only, or preferentially, in a certain portion of the gastrointestinal tract, optionally, in a delayed manner. Examples of embedding compositions that can be used include polymeric substances and waxes. The ER and/or ERR agonist or antagonist can also be in microencapsulated form, if appropriate, with one or more of the above-described excipients.

Liquid dosage forms for oral administration include pharmaceutically acceptable emulsions, microemulsions, solutions, suspensions, syrups and elixirs. In addition to the L-lysine, glucosamine, chondroitin sulfate and/or methylsulfonyl methane, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the L-lysine, glucosamine, chondroitin sulfate and/or methylsulfonyl methane, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for the topical or transdermal administration of L-lysine, glucosamine, chondroitin sulfate and/or methylsulfonyl methane include powders, sprays, ointments, pastes, creams, lotions, gels, solutions, patches and inhalants. The active component may be mixed under sterile conditions with a pharmaceutically acceptable carrier, and with any preservatives, buffers, or propellants that may be required. The ointments, pastes, creams and gels may contain, in addition to the L-lysine, glucosamine, chondroitin sulfate and/or methylsulfonyl methane, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to the L-lysine, glucosamine, chondroitin sulfate and/or methylsulfonyl methane, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

L-lysine, glucosamine, chondroitin sulfate and/or methylsulfonyl methane compositions can be alternatively administered by aerosol. This is accomplished by preparing an aqueous aerosol, liposomal preparation or solid particles containing the L-lysine, glucosamine, chondroitin sulfate and/or methylsulfonyl methane. A nonaqueous (e. g., fluorocarbon propellant) suspension could be used. Sonic nebulizers can also be used. An aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches can also be used to deliver L-lysine, glucosamine, chondroitin sulfate and/or methylsulfonyl methane compositions to the body. Such formulations can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Formulations suitable for parenteral administration comprise a L-lysine, glucosamine, chondroitin sulfate and/or methylsulfonyl methane composition in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacterostats, solutes which render the formulation isotonic with the blood of the intended recipient or suspending or thickening agents.

Examples of suitable aqueous and nonaqueous carriers which may be employed in the formulations suitable for parenteral administration include water, ethanol, polyols (e. g., such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Formulations suitable for parenteral administration may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, or phenol sorbic acid. It may also be desirable to include isotonic agents, such as sugars, sodium chloride, and the like into the compositions. In addition, prolonged absorption of the injectable pharmaceutical form may be brought about by the inclusion of agents, which delay absorption such as aluminum monostearate and gelatin.

In some cases, in order to prolong the effect of a L-lysine, glucosamine, chondroitin sulfate and/or methylsulfonyl methane composition, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered formulation is accomplished by dissolving or suspending the L-lysine, glucosamine, chondroitin sulfate and/or methylsulfonyl methane composition in an oil vehicle.

Injectable depot forms are made by forming microencapsuled matrices of L-lysine, glucosamine, chondroitin sulfate and/or methylsulfonyl methane or in biodegradable polymers such as polylactide-polyglycolide. Depending on the ratio of the L-lysine, glucosamine, chondroitin sulfate and/or methylsulfonyl methane. to polymer, and the nature of the particular polymer employed, the rate of drug release can be controlled. Examples of other biodegradable polymers include poly(orthoesters) and poly(anhydrides). Depot injectable formulations are also prepared by entrapping the L-lysine, glucosamine, chondroitin sulfate and/or methylsulfonyl methane in liposomes or microemulsions that are compatible with body tissue.

### EXAMPLES:

A formulation comprising 1500 mg of L-lysine, 3000 mg of Glucosamine, 1800 mg of Chondroitin Sulfate and 1500 mg of MSM was administered four times daily for approximately five to six weeks to an adult male having patches of dry scale approximately ¼ inch thick. The subject's psoriasis was reduced during that time period from ¼ inch scale to pink healthy skin. After the five to six week treatment period, the subject continued treatment with a maintenance dosage, which was approximately half of the initial treatment dosage. During that time the subject's psoriasis did not return in any significant manner.

A second subject, an adult female having symptoms associated with psoriasis was asked to take the same dosage described above. Even though the subject did not consistently take the above dosages and instead self-administered something less than the recommended dosage, the subject reported reduced irritation during the course of treatment.

The descriptions in the present invention are provided only as examples and should not be understood to be limiting on the claims. Based on the description, a person of ordinary skill in the art may make modifications and changes to the preferred embodiments, which does not depart from the scope of the present invention.

### REFERENCES

1. Griffith R S, Walsh D E, Myrmel K H, et al., Success of L-lysine therapy in frequently recurrent herpes simplex infection, Treatment and prophylaxis, Dermatologica 1987;175:183-190.

2. McCune M A, Perry H O, Muller S A, et al., Treatment of recurrent herpes simplex infections with L-lysine monohydrochloride, Cutis 1984; 34:366-373.

3. Milman N, Scheibel J, Jessen O., Lysine prophylaxis in recurrent herpes simplex labialis: a double-blind, controlled crossover study, Acta Derm Venereol. 1980; 60:85-87.

4. Griffith R S, DeLong D C, Nelson J D, Relation of arginine-lysine antagonism to herpes simplex growth in tissue culture, Chemotherapy, 1981; 27:209-213.

5. Griffith R S, Walsh D E, Myrmel K H, et al. Success of L-lysine therapy in frequently recurrent herpes simplex infection, Treatment and prophylaxis. Dermatologica. 1987; 175:183-190.

6. McCune M A, Perry H O, Muller S A, et al., Treatment of recurrent herpes simplex infections with L-lysine monohydrochloride, Cutis. 1984; 34:366-373.

7. Das A Jr and Hammad T A, Efficacy of a combination of FCHG49 glucosamine hydrochloride, TRH122 low molecular weight sodium chondroitin sulfate and manganese ascorbate in the management of knee osteoarthritis, Osteoarthritis Cartilage, 2000; 8:343- 350.

8. Noack W, Fischer M, Forster K K, et al., Glucosamine sulfate in osteoarthritis of the knee, Osteoarthritis Cartilage, 1994; 2:51-59.

9. Rovati L C., A large, randomized, placebo controlled, double-blind study of glucosamine sulfate vs. piroxicam and vs. their association, on the kinetics of the symptomatic effect in knee osteoarthritis, Osteoarthritis Cartilage, 1994; 2(suppl 1):56.

10. Rovati L C, The practical development of a selective drug for osteoarthritis: glucosamine sulfate, Presented at: The Ninth EULAR Symposium, Oct. 7-10, 1996; Madrid, Spain.

11. Bucsi L, Poor G. Efficacy and tolerability of oral chondroitin sulfate as a symptomatic slow-acting drug for osteoarthritis (SYSADOA) in the treatment of knee osteoarthritis, Osteoarthritis Cartilage, 1998; 6(suppl A):31-36.

12. Conrozier T, Anti-arthrosis treatments: efficacy and tolerance of chondroitin sulfates (CS 4&6) [translated from French], Presse Med. 1998; 27:1862-1865.

14. Lawrence R, Lignisul MSM (methylsulfonylmethane): A double-blind study of its use in degenerative arthritis (unpublished), Available at:

http://www.msm.com/msmresearch.cfm.

15. Jacob S W, The current status on the use of a new pharmacologic approach (dimethyl sulfone) for the relief of snoring, Oregon Health Sciences University. Unpublished.

16. McCabe D, O'Dwyer P, Sickle-Santanello B, et al. Polar solvents in the chemoprevention of dimethylbenzanthracene-induced rat mammary cancer, Arch Surg. 1986; 121:1455-1459.

17. O'Dwyer P J, McCabe D P, Sickle-Santanello B J, et al., Use of polar solvents in chemoprevention of 1,2-dimethylhydrazine-induced colon cancer, Cancer. 1988; 62:944-948

18. Earl L. Mindell, The MSM Miracle—Enhance your health with organic sulfur 1997.

19. Genaro, A. R., Remington: The Science and Practice of Pharmacy 2003.

What is claimed is:

1. A composition for the treatment of psoriasis comprising:
L-lysine;
glucosamine;
chondroitin; and
methylsulfonyl methane.

2. The composition of claim 1 wherein:
the L-lysine comprises at least 10% by weight of the total weight of the L-lysine, glucosamine, chondroitin and methylsulfonyl methane;
the glucosamine comprises at least 30% by weight of the total weight of the L-lysine, glucosamine, chondroitin and methylsulfonyl methane;
the chondroitin comprises at least 15% by weight of the total weight of the L-lysine, glucosamine, chondroitin and methylsulfonyl methane; and
the methylsulfonyl methane comprises at least 10% by weight of the total weight of the L-lysine, glucosamine, chondroitin and methylsulfonyl methane.

3. The composition of claim 1 wherein:
the L-lysine comprises between about 10% and 30% by weight of the total weight of the L-lysine, glucosamine, chondroitin and methylsulfonyl methane;
the glucosamine comprises between about 30% to 50% by weight of the total weight of the L-lysine, glucosamine, chondroitin and methylsulfonyl methane;
the chondroitin comprises between about 15% to 35% by weight of the total weight of the L-lysine, glucosamine, chondroitin and methylsulfonyl methane; and
the methylsulfonyl methane comprises between about 10% to 30% by weight of the total weight of the L-lysine, glucosamine, chondroitin and methylsulfonyl methane.

4. The composition of claim 1 wherein:
the L-lysine comprises between about 15% and 25% by weight of the total weight of the L-lysine, glucosamine, chondroitin and methylsulfonyl methane;
the glucosamine comprises between about 35% to 45% by weight of the total weight of the L-lysine, glucosamine, chondroitin and methylsulfonyl methane;
the chondroitin comprises between about 20% to 30% by weight of the total weight of the L-lysine, glucosamine, chondroitin and methylsulfonyl methane; and
the methylsulfonyl methane comprises between about 15% to 25% by weight of the total weight of the L-lysine, glucosamine, chondroitin and methylsulfonyl methane.

5. The composition of claim 4 further comprising a pharmaceutically acceptable carrier.

6. The composition of claim 5 wherein the composition is formulated for oral delivery.

7. The composition of claim 6 wherein the total weight of the L-lysine, glucosamine, chondroitin and methylsulfonyl methane of the composition is at least about 5000 mg.

8. The composition of claim 6 wherein the total weight of the L-lysine, glucosamine, chondroitin and methylsulfonyl methane of the composition is between about 6000 mg and 10000 mg.

9. The composition of claim 6 wherein the total weight of the L-lysine, glucosamine, chondroitin and methylsulfonyl methane of the composition is between 7000 mg and 9000 mg.

10. A method for the treatment of psoriasis comprising the step of:
orally administering a composition comprising L-lysine, glucosamine, chondroitin, and methylsulfonyl methane.

11. The method of claim 10 wherein a single dosage of the composition to be administered comprises:
L-lysine in a concentration of at least about 10% by weight of the total weight of the L-lysine, glucosamine, chondroitin and methylsulfonyl methane;
glucosamine in a concentration of at least about 30% by weight of the total weight of the L-lysine, glucosamine, chondroitin and methylsulfonyl methane;
chondroitin in a concentration of at least about 15% by weight of the total weight of the L-lysine, glucosamine, chondroitin and methylsulfonyl methane; and
methylsulfonyl methane in a concentration of at least about 10% by weight of the total weight of the L-lysine, glucosamine, chondroitin and methylsulfonyl methane.

12. The method of claim 10 wherein a single dosage of the composition to be administered comprises:
L-lysine in a concentration between about 10% and 30% by weight of the total weight of the L-lysine, glucosamine, chondroitin and methylsulfonyl methane;
glucosamine in a concentration between about 30% to 50% by weight of the total weight of the L-lysine, glucosamine, chondroitin and methylsulfonyl methane;
chondroitin in a concentration between about 15% to 35% by weight of the total weight of the L-lysine, glucosamine, chondroitin and methylsulfonyl methane; and
methylsulfonyl methane in a concentration between about 10% to 30% by weight of the total weight of the L-lysine, glucosamine, chondroitin and methylsulfonyl methane.

13. The method of claim 10 wherein a single dosage of the composition to be administered comprises:
L-lysine in a concentration between about 15% and 25% by weight of the total weight of the L-lysine, glucosamine, chondroitin and methylsulfonyl methane;
glucosamine in a concentration between about 35% to 45% by weight of the total weight of the L-lysine, glucosamine, chondroitin and methylsulfonyl methane;
chondroitin in a concentration between about 20% to 30% by weight of the total weight of the L-lysine, glucosamine, chondroitin and methylsulfonyl methane; and
methylsulfonyl methane in a concentration between about 15% to 25% by weight of the total weight of the L-lysine, glucosamine, chondroitin and methylsulfonyl methane.

14. The method of claim 13 wherein the composition further comprises a pharmaceutically acceptable carrier.

15. The method of claim 13 further comprising the step of administering the dosage at least four times daily.

16. The method of claim 13 wherein the total weight of the L-lysine, glucosamine, chondroitin and methylsulfonyl methane of the composition is at least about 5000 mg.

17. The method of claim 13 wherein the total weight of the L-lysine, glucosamine, chondroitin and methylsulfonyl methane of the composition is between 6000 mg and 10000 mg.

18. The method of claim 13 wherein the total weight of the L-lysine, glucosamine, chondroitin and methylsulfonyl methane of the composition is between 7000 mg and 9000 mg.

19. The method of claim 18 further comprising the step of reducing the total weight of the dosage of L-lysine, glucosamine, chondroitin and methylsulfonyl methane as the symptoms associated with psoriasis diminish.

20. A system for the treatment of psoriasis comprising:
one or more packets of one or more tablets for oral consumption,
wherein the tablets comprise L-lysine, glucosamine, chondroitin and methylsulfonyl methane.

21. The system of claim 20 wherein each of the packets comprise substantially the same contents.

22. The system of claim 13 wherein the total weight of the L-lysine, glucosamine, chondroitin and methylsulfonyl methane of the tablets of each packet is between about 7000 mg and 9000 mg.

23. The system of claim 22 wherein the each packet comprises:
L-lysine in a concentration between about 15% and 25% by weight of the total weight of the L-lysine, glucosamine, chondroitin and methylsulfonyl methane;
glucosamine in a concentration between about 35% to 45% by weight of the total weight of the L-lysine, glucosamine, chondroitin and methylsulfonyl methane;
chondroitin in a concentration between about 20% to 30% by weight of the total weight of the L-lysine, glucosamine, chondroitin and methylsulfonyl methane; and
methylsulfonyl methane in a concentration between about 15% to 25% by weight of the total weight of the L-lysine, glucosamine, chondroitin and methylsulfonyl methane.

24. The system of-claim 23 wherein each packet comprises at least two tablets.

25. The system of claim 24 wherein at least one of the at least two tablets consists essentially of L-lysine.
